(19) United States
(12) Patent Application Publication
(10) Pub. No.: US 2023/0158340 A1
(43) Pub. Date: May 25, 2023

(54) PERSONAL GAS SUPPLY APPARATUS AND METHODS OF USE THEREOF

(71) Applicant: BIOMEDTECH AUSTRALIA PTY LTD, MOORABBIN, VICTORIA (AU)

(72) Inventor: Oleg Bassovitch, MOORABBIN,VICTORIA (AU)

(21) Appl. No.: 17/802,379

(22) PCT Filed: Feb. 24, 2021
(86) PCT No.: PCT/AU2021/050155

§ 371 Date: Aug. 25, 2022

(30) Foreign Application Priority Data
Feb. 26, 2020 (AU) 2020900549

(51) Int. Cl. A62B 7/14; A61M 16/06; A61M 16/10; A61M 16/00; A62B 9/00

(52) U.S. Cl. CPC A62B 7/14; A61M 16/0003; A61M 16/022; A61M 16/0616; A61M 16/1005; A62B 9/00

(57) ABSTRACT

A personal breathing apparatus installed in an aircraft that is not fully pressurized, and configured to prevent or treat an adverse physiological event. The personal breathing system is configured so as to prevent, lessen or reverse hypercapnia by (i) facilitating removal of carbon dioxide generated the pilot by controlling pilot ventilation and/or (ii) limiting or decreasing the amount of carbon dioxide generated by the pilot by controlling the amount of oxygen breathed by the pilot.

### FIELD OF THE INVENTION

. The present invention relates generally to the field of aviation medicine, and particularly military aviation medicine. In one aspect the invention relates to personal breathing apparatus installed in a military aircraft, the apparatus configured to supply oxygen at certain levels and/or extract carbon dioxide from a pilot so as to prevent or treat an adverse physiological event. Another aspect of the invention relates to methods for treating or preventing an unexpected physiological event in a pilot by the administration of certain gas mixtures and/or the extraction of carbon dioxide.

### BACKGROUND TO THE INVENTION

. Pilots and other flight personnel are exposed to environmental conditions that may adversely affect normal physiological functioning of the body. The term “physiological event” is often used in the art to describe the aberrant functioning of the body caused by an environmental condition within an aircraft.

. The result of a physiological event may be catastrophic, especially where a pilot’s cognitive functions are severely impaired or there is a loss of consciousness. The pilot may lose control of the aircraft leading to a crash situation and loss of life.

. In some instances, the cause of a physiological event is unknown, and in which case the term “unexplained physiological event” (or “UPE”) is often used.

. Over the past decade, the United States Navy and United States Marine Corps have noted an alarming increase in the number of UPEs. During 2016, over 120 UPEs occurred in US Naval aircraft utilizing Onboard Oxygen Generating Systems (OBOGS). That number represented an increase of 400% over the preceding 5 years.

. UPEs are reported in association with many aircraft operated by the US Navy and Air Force, including the F-22 Raptor, the F-35A Joint Strike Fighter, the A-10 Thunderbolt, the T-45 Goshawk trainer and F/A-18 Hornet. Because UPEs were associated with a wide variety of aircraft were affected, in seeking to address the problem the services assumed that a variety of problems would need to be solved. In his testimony to the House Armed Services Tactical Air and Land Forces Subcommittee in February of 2018, Air Force deputy chief of staff for operations Lt. Gen. Mark Nowland, stated that “there is no single root cause tied to a manufacturing or design defect that would explain multiple physiological event incidents across airframes or within a specific airframe.”

. In 2010 a F-22 Raptor malfunctioned in mid-flight and was lost along with its pilot. The pilot’s oxygen system was officially blamed for the loss of life and a US$377 M aircraft. However UPEs continued to happen, and the entire fleet of F-22 Raptor was grounded for five months in 2011.

. Secretary of the Air Force Public Affairs published online on Jun. 09, 2017: “In order to synchronize operations and maintenance efforts toward safe flying operations we have cancelled local F-35A flying ...The Air Force takes these physiological incidents seriously, and our focus is on the safety and our focus is on the safety and well-being of our pilots. We are taking the necessary steps to find the root cause of these incidents.”

. Nevertheless, UPEs continued to be problematic and were the cause of repeated grounding the 19th Air Force’s T-6 Texan trainer in 2018.

. In seeking to identify the root cause of UPEs, much research has been devoted to identifying contaminants entering the pilot’s breathing apparatus. In high performance military aircraft, the pressure required to drive gases into the pilot’s breathing mask is typically provided by the aircraft engine. Some prior artisans have sensibly reasoned that combustion products about the engine were contaminating the breathing apparatus and affecting the pilot’s neurological functioning. An alternative hypothesis attributes variation in engine revolutions (and especially where revolutions decrease intermittently at low throttle settings) as causative of suboptimal delivery of gas to the pilot.

. It is an aspect of the present invention to provide an improvement to prior art breathing apparatus, and also methods for treating or preventing UPEs in pilots. A further aspect is to provide a useful alternative to prior art breathing apparatus and methods for treating or preventing UPEs in pilots. Yet a further aspect is to identify a hitherto unknown cause of UPEs and to provide breathing apparatus and methods for treating or preventing UPEs in pilots based on the identified cause.

. The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

. In a first aspect, but not necessarily the broadest aspect, the present invention provides a personal breathing system for use by a pilot in a military aircraft, the system comprising:
(a) a breathing mask capable of forming a seal about the pilot’s mouth at least, the seal being sufficient so as to allow control of pilot ventilation or the amount of oxygen breathed by the pilot, the breathing mask having a microphone associated therewith for voice communications from the pilot,
(b) a source of substantially pure oxygen gas in gaseous communication with the breathing mask, and
(c) a gas delivery regulator configured to (i) control pilot ventilation and/or (ii) limit or decrease the amount of carbon dioxide generated by the pilot by controlling the amount of oxygen gas breathed by the pilot so as to prevent, lessen or reverse hypercapnia.

. In one embodiment of the first aspect, the extent of prevention or reversal of hypercapnia is sufficient so as to prevent, lessen or reverse an adverse physiological event in the pilot.

. In one embodiment of the first aspect, pilot ventilation is controlled so as to increase exit of carbon dioxide from the pilot’s blood into the air spaces of the lungs, the increase in exit being sufficient to prevent, lessen or reverse hypercapnia in the pilot.

. In one embodiment of the first aspect, pilot ventilation is controlled so as to increase expulsion of carbon dioxide from the air spaces in the pilot’s lungs, the increase in expulsion being sufficient to prevent, lessen or reverse hypercapnia in the pilot.

. In one embodiment of the first aspect, pilot ventilation is controlled so as to correct a V/Q ratio mismatch in the pilot (V=ventilation and Q=perfusion).

. In one embodiment of the first aspect, pilot ventilation is controlled to increase the V/Q ratio, the increase being sufficient to prevent, lessen or reverse hypercapnia in the pilot.

. In one embodiment of the first aspect, pilot ventilation is controlled by reference to ventilation rate, ventilation pressure, tidal volume, breathing rate, positive end expiratory pressure, tidal volume, or FiO₂ (fraction of inspired oxygen).

. In one embodiment of the first aspect, ventilation rate is increased, or tidal volume is increased or ventilation pressure is increased, the increase being sufficient to prevent, lessen or reverse hypercapnia in the pilot.

. In one embodiment of the first aspect, the personal breathing system comprises an actuator configured to modulate ventilation of the pilot so as to prevent, lessen or reverse hypercapnia in the pilot.

. In one embodiment of the first aspect, the actuator is manually actuatable or automatically actuatable.

. In one embodiment of the first aspect, the personal breathing system comprises a biofeedback subsystem configured to sense in a pilot a parameter indicative of hypercapnia or likely impending hypercapnia, and to modulate ventilation of the pilot so as to prevent, lessen or reverse hypercapnia in the pilot.

. In one embodiment of the first aspect, the biofeedback subsystem is in operable communication with the actuator, and the system is configured to actuate the actuator so as modulate ventilation of the pilot where hypercapnia is likely impending or detected by the biofeedback subsystem.

. In one embodiment of the first aspect, the personal breathing system comprises oxygen delivery means configured to deliver oxygen to the pilot, and the oxygen delivery means is configured to limit or decrease the amount of carbon dioxide generated by the pilot, the limit or decrease being sufficient so as to prevent, lessen or reverse hypercapnia in the pilot.

. In one embodiment of the first aspect, the personal breathing system is configured to deliver a first breathable gas or gas mixture having a first level of oxygen at a first time point to the pilot , and at a second time point delivering to the pilot a second breathable gas mixture having a second level of oxygen, the second level of oxygen being lower than the first level of oxygen, wherein the system is configured so as to be actuatable so as deliver the second gas mixture so as to prevent, lessen or reverse hypercapnia in the pilot.

. In one embodiment of the first aspect, the first level of oxygen is sufficiently high so as to induce hypercapnia in a pilot breathing the first breathable gas or gas mixture.

. In one embodiment of the first aspect, the first level of oxygen is greater than about 60%, 70%, 80%, 90%, 95%, 96%, 97%, 98%, or 99%.

. In one embodiment of the first aspect, the first level of oxygen is between about 60% and about 100%, between about 70% and about 100%, between about 80% and about 100%, between about 90% and about 100%, between about 95% and about 100%, between about 96% and about 100%, between about 97% and about 100%, between about 98% and about 100%, or between about 99% and about 100%.

. In one embodiment of the first aspect, the second level of oxygen is sufficiently low so as to prevent, lessen or reverse hypercapnia in a pilot having breathed the first gas or gas mixture at the first time point.

. In one embodiment of the first aspect, the second level of oxygen is less that than about 60%, 70%, 80%, 90%, 95%, 96%, 97%, 98%, or 99%.

. In one embodiment of the first aspect, the personal breathing system comprises an actuator configured to effect a decrease the level of oxygen in the breathable gas or gas mixture.

. In one embodiment of the first aspect, the actuator is manually actuatable or automatically actuatable.

. In one embodiment of the first aspect, the personal breathing system comprises a biofeedback subsystem configured to sense in a pilot a parameter indicative of hypercapnia or likely impending hypercapnia, and to lower the level of oxygen delivered to the pilot where hypercapnia likely impending or is detected.

. In one embodiment of the first aspect, the biofeedback subsystem is in operable communication with the actuator, and the system is configured to actuate the actuator so as to lower the level of oxygen delivered to the pilot where hypercapnia or likely impending hypercapnia is detected by the biofeedback subsystem.

. In one embodiment of the first aspect, the personal breathing system comprises a diluent gas source configured to deliver a diluent gas, and system is configured such that oxygen in the first gas or gas mixture is diluted with the diluent gas to provide the second gas mixture.

. In one embodiment of the first aspect, the dilution is sufficient so as to prevent, lessen or reverse hypercapnia in the pilot.

. In one embodiment of the first aspect, the diluent gas has a lower molecular or atomic weight than diatomic nitrogen or diatomic oxygen.

. In one embodiment of the first aspect, the diluent gas is helium.

. In one embodiment of the first aspect, the gas delivery regulator comprises one or more data inputs and the regulator is configured to (i) control pilot ventilation and/or (ii) control the amount of oxygen breathed by the pilot, the control being sufficient so as to prevent, lessen or reverse hypercapnia in the pilot.

. In one embodiment of the first aspect, the personal breathing system comprises one or sensors in data communication with the one or more data inputs of the regulator, and wherein the one or more sensors is/are selected from the group consisting of: a barometer, an altimeter, an accelerometer, an oxygen sensor, a carbon dioxide sensor, and a contaminant sensor.

. In one embodiment of the first aspect, the personal breathing system comprises an electronic controller having program instructions configured to receive input data from the one or more sensors, transform the input data to output data, the output data being in data communication with the regulator, wherein the program instructions are configured to control pilot ventilation and/or the amount of oxygen breathed by the pilot so as to prevent, lessen or reverse hypercapnia in the pilot.

. In one embodiment of the first aspect, the personal breathing system comprises a compressed air source, an oxygen generator as a first possible source of substantially pure oxygen, the oxygen generator having an input in gaseous communication with the compressed air source, and an output through which an oxygen-enriched gas mixture flows, an emergency oxygen source as a second possible source of substantially pure oxygen and/or a recovery gas mix source, the regulator having a first input in gaseous communication with the compressed air source and a second input in gaseous communication with the output of the oxygen generator, and an output, and a third input in gaseous communication with the emergency oxygen source and/or a recovery gas mix source, and the breathing mask is in gaseous communication with the output of the regulator, wherein in use the regulator is configured so as to utilise the compressed air source as (i) a source of diluent gas to dilute oxygen enriched gas output by the oxygen generator, the diluted oxygen enriched gas being conveyed to the breathing mask, or (ii) a source of pressure to deliver pressurized gas or gas mixture to the pilot.

. In a second aspect, the present invention provides a military aircraft having installed thereon the personal breathing system of any embodiment of the first aspect.

. In a third aspect, the present invention provides a method for treating or preventing an adverse physiological event in a pilot disposed in a military aircraft and receiving oxygen supplementation via a breathing mask, the method comprising (i) providing a breathing mask capable of forming a seal about the pilot’s mouth at least, the seal being sufficient so as to allow control of pilot ventilation or the amount of oxygen breathed by the pilot, the breathing mask having a microphone associated therewith for voice communications from the pilot, (ii) facilitating removal of carbon dioxide generated the pilot by controlling pilot ventilation and/or (iii) limiting or decreasing the amount of carbon dioxide generated by the pilot by controlling the amount of oxygen breathed by the pilot, wherein the control of pilot ventilation and/or the control of the amount of oxygen breathed by the pilot is sufficient so as to prevent, lessen or reverse hypercapnia in the pilot.

. In one embodiment of the third aspect, pilot ventilation is controlled so as to increase exit of carbon dioxide from the pilot’s blood into the air spaces of the lungs, the increase in exit being sufficient to prevent, lessen or reverse hypercapnia in the pilot.

. In one embodiment of the third aspect, pilot ventilation is controlled so as to increase expulsion of carbon dioxide from the air spaces in the pilot’s lungs, the increase in expulsion being sufficient to prevent, lessen or reverse hypercapnia in the pilot.

. In one embodiment of the third aspect, pilot ventilation is controlled so as to correct a V/Q ratio mismatch in the pilot (V=ventilation and Q=perfusion).

. In one embodiment of the third aspect, pilot ventilation is controlled to increase the V/Q ratio, the increase being sufficient to prevent, lessen or reverse hypercapnia in the pilot.

. In one embodiment of the third aspect, pilot ventilation is controlled by reference to ventilation rate, ventilation pressure, or tidal volume.

. In one embodiment of the third aspect, ventilation rate is increased, or tidal volume is increased or ventilation pressure is increased, the increase being sufficient to prevent, lessen or reverse hypercapnia in the pilot.

. In one embodiment of the third aspect, the method comprises use of personal breathing system comprising an actuator configured to modulate ventilation of the pilot so as to prevent, lessen or reverse hypercapnia in the pilot.

. In one embodiment of the third aspect, the actuator is actuated by the pilot or an electronic controller.

. In one embodiment of the third aspect, the method comprises use of a biofeedback subsystem configured to sense in a pilot a parameter indicative of hypercapnia or likely impending hypercapnia, and causing or allowing the biofeedback system to modulate ventilation of the pilot so as to prevent, lessen or reverse hypercapnia in the pilot.

. In one embodiment of the third aspect, the biofeedback subsystem is in operable communication with the actuator, and the actuator is actuated so as modulate ventilation of the pilot where hypercapnia is likely impending or detected by the biofeedback subsystem.

. In one embodiment of the third aspect, the method comprises the steps of delivering a first breathable gas or gas mixture having a first level of oxygen at a first time point to the pilot, and at a second time point delivering to the pilot a second breathable gas mixture having a second level of oxygen, the second level of oxygen being lower than the first level of oxygen so as to prevent, lessen or reverse hypercapnia in the pilot.

. In one embodiment of the third aspect, the method comprises the step of the pilot or an electronic controller actuating an actuator to effect delivery of the second gas mixture

. In one embodiment of the third aspect, the first level of oxygen is sufficiently high so as to induce hypercapnia in a pilot breathing the first breathable gas or gas mixture.

. In one embodiment of the third aspect, the first level of oxygen is greater than about 60%, 70%, 80%, 90%, 95%, 96%, 97%, 98%, or 99%.

. In one embodiment of the third aspect, the first level of oxygen is between about 60% and about 100%, between about 70% and about 100%, between about 80% and about 100%, between about 90% and about 100%, between about 95% and about 100%, between about 96% and about 100%, between about 97% and about 100%, between about 98% and about 100%, or between about 99% and about 100%.

. In one embodiment of the third aspect, the second level of oxygen is sufficiently low so as to prevent, lessen or reverse hypercapnia in a pilot having breathed the first gas or gas mixture at the first time point.

. In one embodiment of the third aspect, the second level of oxygen is less that than about 60%, 70%, 80%, 90%, 95%, 96%, 97%, 98%, or 99%.

. In one embodiment of the third aspect, the method comprises use of an actuator configured to effect a decrease the level of oxygen in the breathable gas or gas mixture.

. In one embodiment of the third aspect, the actuator is manually actuatable or automatically actuatable.

. In one embodiment of the third aspect, the method comprises a biofeedback subsystem configured to sense in a pilot a parameter indicative of hypercapnia or likely impending hypercapnia, and to lower the level of oxygen delivered to the pilot where hypercapnia likely impending or is detected.

. In one embodiment of the third aspect, the biofeedback subsystem is in operable communication with the actuator, and the biofeedback subsystem actuates the actuator so as to lower the level of oxygen delivered to the pilot where hypercapnia or likely impending hypercapnia is detected by the biofeedback subsystem.

. In one embodiment of the third aspect, the method comprises use of a diluent gas source configured to deliver a diluent gas, and the method comprises diluting the oxygen in the first gas or gas mixture with the diluent gas to provide the second gas mixture.

. In one embodiment of the third aspect, the dilution is sufficient so as to prevent, lessen or reverse hypercapnia in the pilot.

. In one embodiment of the third aspect, the diluent gas has a lower molecular or atomic weight than diatomic nitrogen or diatomic oxygen.

. In one embodiment of the third aspect, the diluent gas is helium.

. In one embodiment of the third aspect, the method comprises use of a gas delivery regulator and the gas delivery regulator (i) controls pilot ventilation and/or (ii) limits or decreases the amount of carbon dioxide generated by the pilot by controlling the amount of oxygen breathed by the pilot, either on its own or in combination with one or more other components.

. In one embodiment of the third aspect, the gas delivery regulator comprises one or more data inputs and the regulator is configured to (i) control pilot ventilation and/or (ii) control the amount of oxygen breathed by the pilot, the control being sufficient so as to prevent, lessen or reverse hypercapnia in the pilot.

. In one embodiment of the third aspect, the method comprises use of one or sensors in data communication with the one or more data inputs of the regulator, and wherein the one or more sensors is/are selected from the group consisting of: a barometer, an altimeter, an accelerometer, an oxygen sensor, a carbon dioxide sensor, and a contaminant sensor.

. In one embodiment of the third aspect, the method comprises use of an electronic controller having program instructions receiving input data from the one or more sensors, transforming the input data to output data, the output data being communicated to the regulator, wherein the program instructions are control pilot ventilation and/or the amount of oxygen breathed by the pilot so as to prevent, lessen or reverse hypercapnia in the pilot.

. In one embodiment of the third aspect, the method comprises: use of a compressed air source, use of an oxygen generator having an input in gaseous communication with the compressed air source, and an output through which an oxygen-enriched gas mixture flows, use of an emergency oxygen source and/or a recovery gas mix source, use of a regulator having a first input in gaseous communication with the compressed air source and a second input in gaseous communication with the output of the oxygen generator, and an output, and a third input in gaseous communication with the emergency oxygen source and/or a recovery gas mix source, and use of a breathing mask configured to be worn by a pilot, the mask in gaseous communication with the output of the regulator, wherein the regulator utilises the compressed air source as (i) a source of diluent gas to dilute oxygen enriched gas output by the oxygen generator, the diluted oxygen enriched gas is conveyed to the breathing mask, or (ii) a source of pressure to deliver pressurized gas or gas mixture to the pilot.

. In one embodiment of the third aspect, the method is performed in a military aircraft on a pilot therein.

. In one embodiment of the third aspect, the aircraft at a first time point is on the ground, and a second time point at an altitude requiring oxygen supplementation for the pilot, and the step of decreasing the level of oxygen breathed by the pilot occurs at the altitude requiring oxygen supplementation for the pilot.

. In one embodiment of the third aspect, after the step of decreasing the level of oxygen breathed by the pilot occurs at the altitude requiring oxygen supplementation for the pilot.

. In one embodiment of the third aspect, the method comprises the step of the aircraft returning to the ground.

. In a fourth aspect, the present invention provides a method of training a military aircraft pilot to recognize a current or impending physiological event, the method comprising the step of exposing the subject to conditions to induce hypercapnia in the subject, the step of exposing occurring in the course of the subject performing a flight simulation activity.

. In one embodiment of the fourth aspect, the flight simulation activity is performed by way of a computer flight simulator.

### BRIEF DESCRIPTION OF THE FIGURES

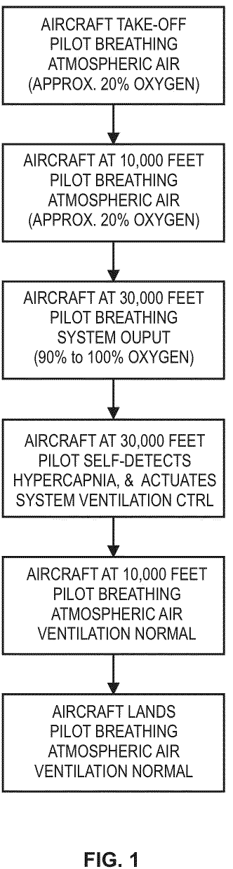

. FIG. 1 is a flowchart illustrating the sequential steps in an exemplary method of the invention utilising a manually actuatable breathing system operable by the pilot to control ventilation upon self-detection of hypercapnia or an adverse physiological event so as to facilitate washout of carbon dioxide form the pilot’s lungs and in turn from the pilot’s blood. (“Art = arterial”)

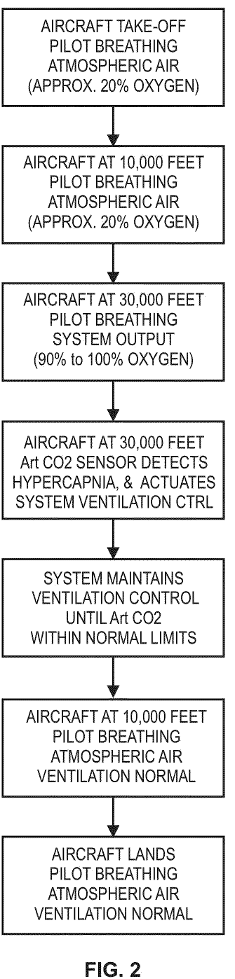

. FIG. 2 is a flowchart illustrating the sequential steps in an exemplary method of the invention utilising an automatically actuatable breathing system configured to detect hypercapnia, and automatically control pilot ventilation to facilitate washout of carbon dioxide. (“Art = arterial”)

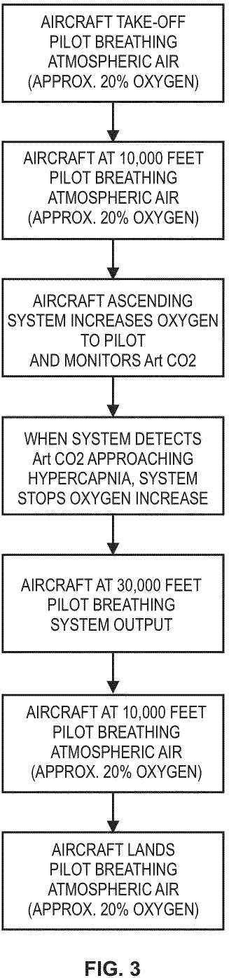

. FIG. 3 is a flowchart illustrating the sequential steps in an exemplary method of the invention utilising an automatically actuatable breathing system configured to increase oxygen delivery automatically, but cease further increases to avoid the pilot from suffering hypercapnia. (“Art = arterial”)

. FIG. 4 is a block diagram illustrating a prior art breathing system having an oxygen generator and an emergency oxygen supply.

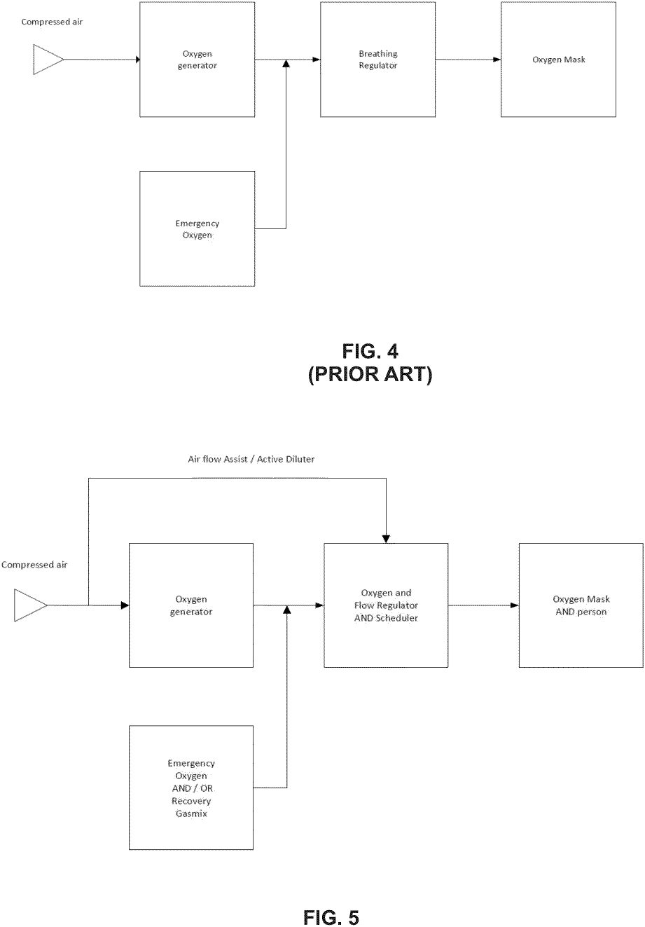

. FIG. 5 is a block diagram illustrating an exemplary breathing system of the present invention configured to lower the level of oxygen in a gas mixture delivered to a pilot.

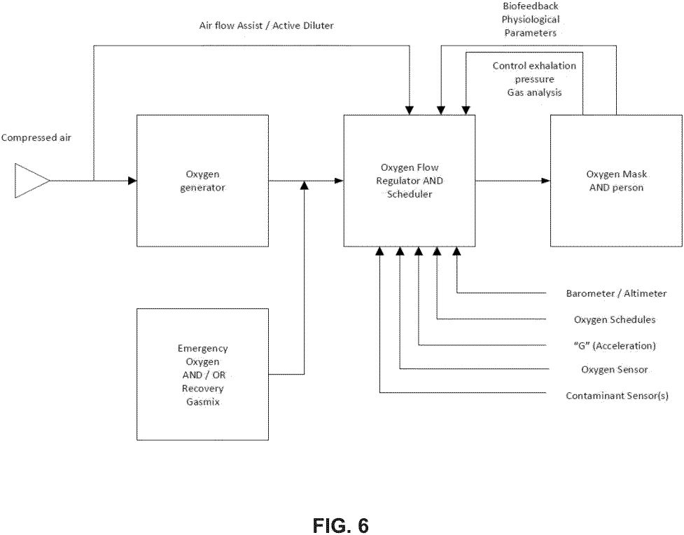

. FIG. 6 is a block diagram illustrating an exemplary breathing system of the present invention configured to lower the level of oxygen in a gas mixture delivered to a pilot, and a biofeedback system and various environmental inputs.

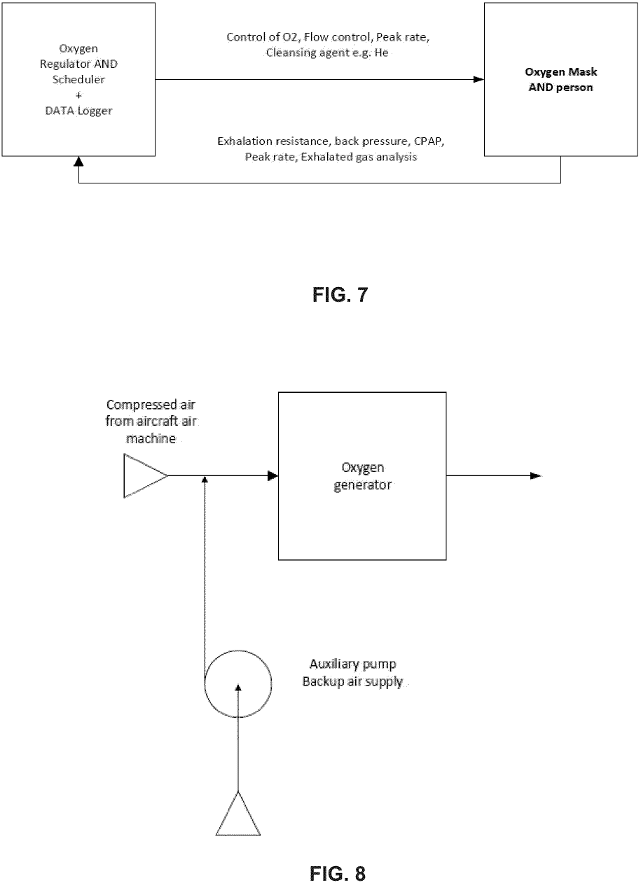

. FIG. 7 is a block diagram illustrating an exemplary feedback loop operable in a breathing system of the present invention configured to sense output parameters of exhalation and exhaled breath, and to use same to modulate oxygen level, gas flow control, peak rate control and diluent gas.

. FIG. 8 is a block diagram of a portion of the system illustrated in FIG. 5, comprising an auxiliary pump and backup air supply for use in the event of low or non-existent compressed air provided by the aircraft.

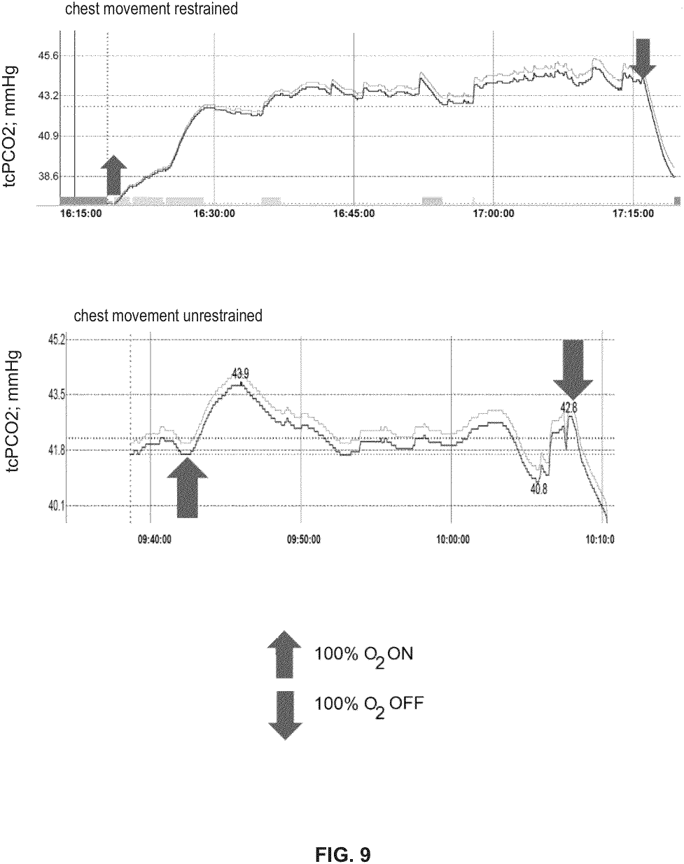

. FIG. 9 is a graph showing the effect of chest restraint versus no restraint for PCO2 in a subject breathing 100% oxygen.

. The drawings are not prepared to any particular scale or dimension and are not presented as being a completely accurate presentation of the various embodiments.

### DETAILED DESCRIPTION OF THE INVENTION AND PREFERRED EMBODIMENTS THEREOF

. After considering this description it will be apparent to one skilled in the art how the invention is implemented in various alternative embodiments and alternative applications. However, although various embodiments of the present invention will be described herein, it is understood that these embodiments are presented by way of example only, and not limitation. As such, this description of various alternative embodiments should not be construed to limit the scope or breadth of the present invention. Furthermore, statements of advantages or other aspects apply to specific exemplary embodiments, and not necessarily to all embodiments, or indeed any embodiment covered by the claims.

. Throughout the description and the claims of this specification the word “comprise” and variations of the word, such as “comprising” and “comprises” is not intended to exclude other additives, components, integers or steps.

. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may.

. The present invention is predicated at least in part on the proposal that a UPE may be the result of a pilot receiving excess oxygen from an OBOGS, the excess oxygen leading to hypercapnia in the pilot. To the best of Applicant’s knowledge, this cause has never before been considered by prior artisans as a cause of UPEs. As discussed in the Background section herein, research into the cause of UPEs has concentrated on the aircraft and breathing equipment. The present inventor has diverted from those established lines of enquiry and carefully considered a cause originating in the pilot’s own respiratory system.

. It is dogma in the art of aviation medicine that hyperoxic gas mixtures (i.e. being enriched in oxygen compared to the atmosphere at sea level) are provided to a pilot from an altitude of around 10,000 feet, with the level of oxygen in the mixture being increased as the aircraft increases altitude. It is further dogma in the art that at relatively high altitudes (say, over 30,000 feet) a pilot is provided with virtually pure oxygen gas. At over 40,000 feet virtually pure oxygen gas is provided to the pilot at pressure. Moreover, prior art emergency oxygen systems provide virtually pure oxygen to the pilot when activated. Against the background of the art-accepted view that oxygen is universally beneficial, the present inventor has discovered that excessive levels of oxygen delivered to a pilot at altitude can lead to hypercapnia.

. More specifically, it is proposed that hypoventilation is caused by suppression of the hypoxic ventilatory drive (mediated by the carotid body and aortic body centres) resulting from hypercapnic respiratory drive suppression. The hypercapnic respiratory drive suppression may be due to a lack of available breathable gas, breath holding (a habit particular to some pilots), obstruction to breathing caused by the resistance of valves in the breathing system, or the weight of flight gear and parachute gear bearing on the pilot’s chest. The resultant hypercapnia results in CO₂ narcosis which may manifest as neurological and sensory effects such as drowsiness, dizziness, confusion, headache, loss of consciousness, vision impairment, hearing impairment, muscle tremor, sweating, shortness of breath and increased heart rate. Such effects in a pilot may be noted separately or seen together in various combinations. These effects are reported by pilots having an UPE, however the underlying cause has always been considered to be hypoxia (i.e. a lack of sufficient oxygen).

. Hypercapnia is basically an abnormally high level of carbon dioxide in the blood, and is seen in patients having an underlying respiratory condition, or where a person is forced to breathe gases having relatively high levels of carbon dioxide. A pilot will typically not have any underlying naturally occurring respiratory condition, and his/her breathing mask does not deliver carbon dioxide rich gas mixture (in fact, the gas mixture is devoid of or has relatively low levels of carbon dioxide). Nevertheless, according to the present invention pilots exhibit symptoms of hypercapnia, and it is proposed that hypercapnia is the cause of at least some UPEs.

. Without wishing to be limited by theory in any way, hypercapnia may be caused by any one or more of the following three potentially interrelated mechanisms:
Impaired V/Q ratio (ventilation / perfusion) caused by atelectasis (i.e. a collapse or closure of all or part of a lung resulting in compromised gas exchange). In aviation, atelectasis can result from excessive acceleration forces occasioned on the pilot’s thoracic region from acceleration or certain manoeuvres, and may be exacerbated by an anti-G suit. In addition or alternatively, an impaired V/Q ratio may be caused by mask dead space which allows for some exhaled carbon dioxide-rich gas to be rebreathed thereby limiting the amount of oxygen that can be used to ventilate the lungs.
The Haldane effect (inefficient CO₂ excretion from tissues)
Hypoventilation (e.g. insufficient oxygen flowrate from an oxygen regulator that force pilots to “save” on inhalations gradually suppressing their hypercapnic drive; weight of flight gear; and resistance to inhalation)

. Of course, UPEs do not occur in all instances of a pilot breathing hyperoxic gases. Factors which inhibit normal ventilation of the lungs may be contributory, such as G-forces occasioned on the pilot, compression of the chest by heavy garments, some pilots being less efficient ventilators than others, tiredness/fatigue, chest wall deformities, weakness in the diaphragm muscles, respiratory infection, administration of some medications (such as analgesics), and metabolic processes that increase carbon dioxide production (such as thyrotoxicosis, increased catabolism form sepsis or hormone imbalance, overfeeding, metabolic acidosis). Furthermore, some individuals have an inherent predisposition for retaining carbon dioxide (so-called “carbon dioxide retainers”). Whilst any of the aforementioned contributory factors may occur at low incidence in a population of pilots, that would explain the relative low frequency of UPEs.

. Furthermore, conditions aboard an aircraft which contribute to UPEs do not occur on all flights. For example, executing certain manoeuvres which cause significant g-forces on a pilot may cause compression of the lungs and/or difficulty in inhaling which may in turn lead to hypoventilation and in turn hypercapnia.

. Identifying the true cause of at least some UPEs allows for new personal breathing systems for pilots, methods of treating and preventing UPEs in a pilot, and methods for training pilots how to detect and overcome UPEs.

. As used herein, the term “adverse physiological event” may be defined by reference to any physiological event which diminishes the pilot’s ability to operate an aircraft. The term is used in preference to UPE (unexplained physiological event) in some contexts where a physiological event is not unexplained and is proposed to be the result of hypercapnia. The physiological event may be defined by reference to a sign or symptom of the pilot such as cognitive deficiency, drowsiness, confusion, visual or auditory disturbance, extended reaction times, loss of consciousness etc. The term “hypercapnia” as used herein may be considered by reference to a measurable physiological parameters, such as a PaCO₂ and in the case of hypercapnia may be elevated above the accepted normal reference value of 45 mm Hg.

. With regards to personal breathing systems, it will be understood that such systems may comprise any one of more of a breathing mask (optionally having inbuilt microphone for radio communications), some means for regulation of gas flows, a hyperoxic gas generator (such as an oxygen concentrator), a compressed oxygen cylinder, means to pressurize gas (if required), and gas tight conduit. An exemplary system is based on an OBOGS, of the type used in military aircraft such as fighter jets, and also for emergency use in transport aircraft.

. In the present invention, the personal breathing system may be configured to improve ventilation parameters of a pilot. Hypercapnia may be prevented or treated by “washing out” of carbon dioxide from the blood. This may be effected by controlling ventilation parameters including ventilation rate, so as to increase the V/Q ratio which in turn increases exit of CO₂ from the blood and across the alveolar wall and into the air space of the lungs. More rapid ventilation may more rapidly expel CO₂ that accumulates in the lungs. Decreasing the partial pressure of CO₂ in the lung air spaces allow for an equilibrium shift to favor the exit of CO₂ from the alveolar blood and into the lung air spaces. Thus, CO₂ more efficiently and continuously exits from the blood thereby preventing or treating hypercapnia, and in turn prevents or treats the symptoms of CO₂ narcosis in the pilot.

. Ventilation rate may be increased by positive pressure ventilation via a breathing mask. Gas is forced into the lungs for a short period after which the pilot is allowed to exhale (actively or passively), and the lungs are again refilled under pressure, and so on in a cyclical manner. The cycle rate (and therefore ventilation rate) can be dictated by the rate at which gas is forced into the lungs. This is achievable by, for example, using a pressurized source of breathable gas and cyclically connecting and disconnecting the pressurized gas to the pilot’s breathing mask. In addition or alternatively, the system may be configured to issue an audible, visual, tactile or haptic alarm instructing the pilot to increase his/her breathing rate. In addition or alternatively, the system may monitor the pilot’s breathing (for example using a pressure transducer or flow meter in the breathing circuit) and trigger an inhalation cycle where the pilot has not inhaled for a predetermined period of time.

. Another ventilation parameter is tidal volume. Low tidal volumes may manifest as shallow breathing in a pilot, thereby limiting the turnover of gas in the lung air spaces (and especially the air spaces deeper within the lungs). CO₂ accumulation in the lung is encouraged under conditions of low tidal volume, and accordingly CO₂ tends to remain in the alveolar blood. Increasing tidal volume improves the turnover of gas in the lungs, and therefore increases the rate of expulsion of CO₂. This in turn lowers the partial pressure of CO₂ in the lung air spaces, with equilibrium principles favoring exit of CO₂ from the blood into the air spaces. Again, hypercapnia and symptoms of CO₂ narcosis are prevented or reversed.

. Tidal volume can be increased by delivering breathable gas to the pilot at pressure, resulting in period of “forced” breathing. The breathable gas may be delivered at such a pressure that the lungs are forced to open to a greater extent than before, thereby improving flow of gases into and out of the deeper air spaces of the lungs. In addition or alternatively, the system may be configured to issue an audible, visual, tactile or haptic alarm instructing the pilot to breathe more deeply and/or forcibly exhale with each breath.

. In some instances, it will be preferable to increase both ventilation rate and tidal volume. Higher ventilation rates tend to result in more shallow breathing, and therefore a relatively low tidal volume. In such cases increasing ventilation pressure may prevent any shallowness in breathing

. In some embodiments, increasing ventilation pressure may be used alone, and without deliberately altering ventilation rate or tidal volume. Of course, ventilation pressure may in itself affect ventilation rate or tidal volume, however such effects need not be the primary reason for altering ventilation pressure. As one example, a breathing gas mixture may be supplied to the pilot via a breathing mask via unassisted positive pressure at 30 mmHg (4 kPa). Ventilation pressure may be used alone or in combination with the anti-G-straining maneuver (AGSM). The AGSM consists of a first breathing component whereby the pilot takes rapid (< 1 sec) exhalation/inspiration cycles every 3 seconds. This decreases the level of carbon dioxide in blood, while lessening pressure on the chest and facilitation venous return. The second component of the AGSM is flexion of skeletal muscles of legs and abdomen, thereby constricting blood vessels in the lower body to further assist venous return. It is advantageous to facilitate the return of carbon dioxide rich blood from the lower body to the heart since such blood is directed into the pulmonary circulation to improve perfusion of the lungs and potentially correct any V/Q mismatch.

. The time period for which ventilation parameter(s) are controlled will typically be sufficient until the symptoms of carbon dioxide narcosis are prevented, lessened or reversed. Where the pilot has actually experienced carbon dioxide narcosis, and he/she has returned to a normal physiological state by the present systems or methods, it would be typical for a mission to be terminated and the aircraft returned to base. However, in some circumstances the mission may be continued and the personal breathing apparatus returned to normal operation for an extended period of time.

. In some embodiments of the invention, the system or method is configured to limit or decrease the level of oxygen delivered to a pilot to control the level of oxygen delivered so as to prevent, lessen or reverse hypercapnia, and therefore also prevent, lessen or reverse the effects of carbon dioxide narcosis.

. Without wishing to limited by theory, it is further proposed that controlling oxygen delivery to the pilot has a follow-on effect on brain function. Particularly, hypercapnia causes cerebral vasodilation and where the pilot is exposed to high levels of oxygen the blood carries abnormally high amounts of oxygen to the brain. Excess oxygen markedly increases the rate of generation of ATP from ADP in neural cells, which in turn leads to a glutamate excitotoxicity in the brain. The resultant “glutamatergic storm” may cause the seizures and loss of consciousness reported in some UPEs. Accordingly, in some embodiments of the invention, the level of oxygen delivered to the pilot may be limited or decreased so as to prevent, lessen or reverse any one of more of cerebral vasodilation, generation of abnormally high levels of ATP in the brain, glutamate excitotoxicity, a seizure or loss of consciousness.

. In a preventative approach, the level of oxygen delivered to the pilot may be set at an upper limit below 100% so as to prevent or limit hypercapnia. In the prior art, pure oxygen is delivered unquestionably to a pilot. However, the present inventor has recognized that this approach may encourage hypercapnia and carbon dioxide narcosis by increasing the level of carbon dioxide generated by cellular respiration. It has also been recognized that a pilot does not necessarily require high levels of oxygen when at altitude, and accordingly in many circumstances a pilot will not suffer any adverse consequence where a limitation in oxygen is imposed.

. As is typical in prior art breathing systems, the present system may be configured to increase the level of oxygen delivered to the pilot according to altitude. Thus, at ground level, the pilot may breathe air (i.e. a normoxic gas mixture) with the level of oxygen rising in a step-wise manner as the aircraft passes through certain altitudes so as to avoid hypoxia. Typically, the system comprises an on-board oxygen concentrator which (possibly in combination with a diluent gas) modulate the level of oxygen delivered to the pilot. In the present system, the level of oxygen delivered may be fixed at an upper limit (for example, about 60%) so as to prevent hypercapnia or an adverse physiological event. The figure of about 60% oxygen is proposed to be relevant because if exceeded oxygen-induced atelectasis results, which in turn may lead to hypercapnia. In other embodiments, the system may be configured to deliver oxygen in an amount that induces hypercapnia or an adverse physiological event (such as virtually pure oxygen), however the system is actuatable that where hypercapnia or an adverse physiological event eventuates the level of oxygen is decreased (manually by the pilot or automatically via an electronic control system) to level such that the hypercapnia or adverse physiological event is at least partially reversed.

. The personal breathing system of the present invention may be configured to prevent, lessen or reverse hypercapnia by improving the participation of alveoli in the pilot’s lungs. Improving alveolar participation increases the lung surface area across which carbon dioxide can exit from blood perfusing the lungs. Participation of alveoli can be improved by the application of positive pressure gas via the pilot’s breathing mask at the end of mechanical or spontaneous exhalation. In this way, the pilot’s airway pressure is maintained above atmospheric pressure (as dictated by altitude) so as to oppose passive emptying of the lungs. The pressure opposing passive emptying may be achieved by maintaining a positive pressure flow into the lungs at the end of exhalation (by way of the present personal breathing system), so as to keep the lung air spaces open and ensure that more alveoli are actively participating in gas exchange so as to facilitate carbon dioxide removal and reversal hypercapnia. The positive pressure may be in the region 15 cmH₂O, although may be lower at altitude. The use of positive pressure in this way may be augmented by increasing inspiratory pressure (also by way of the present breathing system) to improve tidal volume. Inspiratory pressures of around 40 cmH₂0 may be used (possibly less at altitude) or to achieve a tidal volume of around 18 ml/kg of body weight. Pressure may be applied at the end of exhalation (and optionally also the applied inspiratory pressure) may be maintained for 10 cycles of inspiration/exhalation or longer as required to address any hypercapnia.

. As an example of manual system actuation, the pilot may be trained to self-detect hypercapnia or an adverse physiological event, and by operating a hand-operable device to cause the system to control ventilation and/or the level of oxygen delivered to the pilot.

. As an example of an automatic system, some sensing means may be incorporated to detect an adverse physiological effect (for example eyes closing, unusual eyeball movement as detected by eye tracking technology, muscle tremors, head dropping) or a physiological parameter associated with acute hypercapnia such as blood carbon dioxide level. With regard to the latter, transcutaneous methods of carbon dioxide analysis are known in the art using heated electrochemical sensors applied to the skin. Such sensors output a continuous estimation of the arterial CO₂ value. Optical sensors are known which operate on the absorption of the near-infrared light, in the evanescent wave of a waveguide integrated in the sensor surface, or in a micro-optics sampling cell.

. A potentially useful commercially available transcutaneous sensor for monitoring blood gases is the V-Sign™ sensor (SenTec AG, Switzerland). This is a Stow-Severinghaus-type PCO₂ sensor combined with 2-wavelength reflectance pulse oximetry. The sensor head comprises a micro pH-electrode and an optical oximetry unit. All data is digitized in the sensor head, allowing the transmission of low-noise signals to a controller.

. A less accurate but potentially useful means of detecting hypercapnia is to detect the level of carbon dioxide in the pilot’s exhaled air.

. In any event when hypercapnia is sensed, the system is triggered to control ventilation and/or the level of oxygen being delivered. In some embodiments, the sensor forms part of a biofeedback system allowing for a ventilation parameter or oxygen level to be varied so as to return within a target range between hypercapnia and hypocapnia. In some embodiments a ventilation parameter and/or the oxygen is varied upwardly and downwardly so as to ensure arterial CO₂ remains within a target range between hypercapnia and hypocapnia.

. In some embodiments of the present system, the level of oxygen in gas delivered to the pilot is modulated by the mixing varying amounts of a diluent gas with a fixed amount of a hyperoxic gas or pure oxygen gas so as to provide a target oxygen level. The diluent gas may be simply atmospheric air. Alternatively, the diluent gas may have a density lower than atmospheric air, or at least lower than diatomic nitrogen gas or diatomic oxygen gas.

. In one embodiment, the diluent gas is helium (i.e. diatomic helium gas) and in which case the helium gas may essentially substitute for nitrogen gas. Nitrogen gas and helium gas have comparable viscosities, but helium has a higher thermal conductivity than nitrogen. Thus a helium/oxygen mixture has a viscosity similar to, but a density lower than a nitrogen/oxygen mixture. Inhalation of the lower density helium/oxygen mixture results in lower turbulence (especially deeper within the lungs), that leading in turn to increased laminar flow and therefore lower airway resistance. Thus, the pilot is able to expel excess carbon dioxide more rapidly so as to return to arterial blood carbon dioxide to normal levels, thereby reversing the hypercapnia and/or the symptoms of an adverse physiological event.

. The diluent gas may function to assist in the removal of carbon dioxide from the lungs, and in turn from the blood. In that regard, a low molecular gas such as helium exhibits less turbulent flow and/or more laminar flow into and out of the smaller air spaces of the lungs. Such gases therefore more readily penetrate into and exit from the deeper regions of the lungs so as to more effective “washout” carbon dioxide from the lungs. Decreasing the partial pressure of carbon dioxide in the lungs favours exit of dissolved carbon dioxide from the blood and into the lung air spaces as a gas.

. Overall control of the system may be by way of microcontroller having inputs receiving environment parameters and/or pilot parameters, and outputs which direct various components (such as oxygen concentrators, regulators, mixers, valves, pumps and the like). Program instructions will typically be provided such that the inputs translate into outputs with the end result that the system prevents or reverses an adverse physiological effect or hypercapnia in the pilot by the close control of ventilation and gases delivered to the pilot.

. With respect to methods of treating or preventing hypercapnia or an adverse physiological event, it will be understood that operationally the step of controlling ventilation and/or the level of oxygen delivered to the pilot occurs at an altitude where the pilot requires supplemental oxygen. Such altitude may be greater than 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000 or 40000 feet. Especially at higher altitudes the pilot will typically be breathing substantially pure oxygen and the danger of hypercapnia significantly increased.

. The level of oxygen may be lowered to a predetermined “safe” level where hypercapnia or the adverse physiological event is reversed. Such a safe level may be 20%, 30%, 40%, 50%, or 60%. The safe level should be chosen such that the pilot does not immediately pass into a hypoxic state, but nevertheless rapidly reverse the hypercapnia or the effects of the adverse physiological event Alternatively, where the system comprises a biofeedback loop the level of oxygen is decreased until a sensed parameter (such as arterial CO₂) is within the normal range and at which time the system stabilizes the level of oxygen in the gas mixture breathed by the pilot.

. The control of ventilation and/or oxygen level may be performed during or shortly after a physical effect on the pilot’s body which causes a V/Q mismatch to occur. In terms of Q (perfusion) the mismatch may be due to decreased blood flow to the lungs as a result of G-forces exerted on the pilot during a maneuver whereby blood tends to pool in the lower extremities and venous return is impaired. In such a circumstance, carbon dioxide rich blood is less able to return to the alveoli to give up carbon dioxide.

. In terms of V (ventilation) the mismatch may be due to a decrease in ventilation such that even if carbon-dioxide rich blood is returned to the lungs, the presence of significant amounts of carbon dioxide remaining stagnant in the alveoli inhibits the exist of the gas into the airspace due to equilibrium principles. Decreases in ventilation may be caused by any one or more of and any combination of: excessive g-forces, a reduction in lung vital capacity resulting from g-induced atelectasis, the pilot being supplied with excessive amounts of oxygen (for example, amounts that result in oxygen-induced atelectasis), the weight of a flight suit and equipment worn about the chest.

. With regards to methods of the invention, the personal breathing systems described herein may be used in isolation or alternatively with various breathing techniques that may be implemented by the pilot to facilitate washing out of carbon dioxide from the lungs. In some embodiments, the personal breathing system may comprise audio, visual, tactile or haptic output to instruct the pilot to perform a particular breathing technique based on a sensed physiological parameter of the pilot or an environment parameter such a g-force, acceleration or altitude. As described infra, AGSM may be used to assist removal of carbon dioxide.

. The present invention may be implemented at ground level for the purpose of training a subject (who may be a pilot or a candidate pilot) to recognize the effects of hypercapnia. The training method may further be used to instruct the subject how to actuate an actuator of a simulated aircraft breathing system so as to control ventilation and/or the level of oxygen delivered. The subject may utilize such training to practice breathing techniques such as forced exhalation in order to assist in the washout of carbon dioxide from the lungs. In some embodiments, the training method may be performed while the subject is operating a flight simulator such that the subject is able to assess the effect of hypercapnia on his/her flying abilities.

. Turning now to the embodiments of the drawings and referring firstly to FIG. 1, the flowchart illustrates a method utilising a manually actuatable breathing system to control pilot ventilation upon self-detection of hypercapnia or an adverse physiological event by the pilot. The system increases the level of oxygen delivered to the pilot while the aircraft climbs, and (as typical in the prior art) when the aircraft is at sufficient altitude, the breathing system delivery pure oxygen of a highly hyperoxic gas mix to the pilot to combat the hypoxia that would otherwise be experienced at that altitude. In this method, the level of oxygen delivered to the pilot is sufficient (possibly in combination with contributing factors such as excessive g-forces) to cause hypercapnia. The pilot is trained in self-detection of hypercapnia and recognises same by the effects on his/her flying ability and other physiological/neurological indicators. The pilot actuates a switch which causes the breathing system to immediately increase ventilation rate and tidal volume so as to rapidly washout carbon dioxide from the blood. The pilot also performs a forced exhalation breathing technique to facilitate carbon dioxide removal from the lungs. Upon reversal of the adverse physiological event, the pilot regains full control of the aircraft and descends to 10,000 feet at which altitude the system reverts to delivering atmospheric oxygen to the pilot and ceases control of ventilation. The aircraft is then landed by the pilot without incident. In the absence of the present system, the pilot would have no means nor motivation to alter ventilation so as to reverse the hypercapnia.

. Referring to the method outlined by the flow chart of FIG. 2, a similar method to that of FIG. 1 is followed although the system comprises an automatically actuatable breathing system configured to detect hypercapnia (by any means), and automatically control pilot ventilation to facilitate washout of carbon dioxide. For example, the hypercapnia may be sensed by a transcutaneous device which transmits an analogue or digital signal to a microcontroller which is programmed to receive the input and convert the input to an output which acts on a valve to mix a diluent gas with an oxygen concentrator output so as to decrease the level of oxygen delivered to the pilot.

. The method of FIG. 3 is a variation on that defined in FIG. 2, in that oxygen level is titrated upwardly during ascent of the aircraft such that the pilot does not approach a hypercapnic state. Where the pilot experiences hypercapnia after cruising altitude is reached or later in the flight, the system carbon dioxide senses that condition and directs the mixing of a diluent gas so as to immediately decrease the level of oxygen delivered to the pilot.

. FIG. 4 is a block diagram illustrating a prior art breathing system having an oxygen generator and an emergency oxygen supply. The system is driven by compressed air drawn from the aircraft engine. The system has an oxygen generator (concentrator) which feeds a regulator which in turn feeds the pilot’s breathing mask. The emergency oxygen supply feeds into the regulator, but is kept isolated therefrom until such a time that emergency oxygen is needed (such as when the pilot considers he is becoming hypoxic) at which time the pilot pulls a ring (not drawn) which acts to connect the emergency oxygen supply to the regulator and ultimately the mask. This arrangement evidences well the central dogmas in the art of aviation medicine that only hypoxia is a problem, and that in a breathing emergency the only gas needed is pure oxygen.

. As shown in the arrangement of FIG. 5 (being a preferred breathing system of the present invention), the compressed air source may be routed to the regulator so as to dilute the high oxygen output of the oxygen generator and lower the level of oxygen delivered to the pilot. The compressed air source may be further used to provide pressure to force gas into the lungs of the pilot so as to improve ventilation and facilitate washout of carbon dioxide from the lungs. This approach is very different that of FIG. 4 which can only provide highly hyperoxic gases to the pilot (there being no means by which the level of oxygen may be lowered) and which is unable to control pilot ventilation. The system of FIG. 5 may nevertheless include emergency oxygen where hypoxia may dictate the use of same.

. A further mechanism to facilitate carbon dioxide washout and lower oxygen levels is shown at FIG. 5, where a recovery gas mixture including helium is used. The mixture may have a “safe” level of oxygen (say 40%) which could be delivered to the pilot to counteract an episode of hypercapnia caused at least part by a build up of carbon dioxide due to a V/Q mismatch, and possibly also the earlier delivery of a high oxygen content gas mixture. The system may configured such that the pilot actuates an actuator that causes mixing of diluent gas at an elevated pressure to improve lung ventilation, or connection of the recovery gas to the regulator. Alternatively, those actions may be triggered automatically by a microcontroller based on inputs indicative of hypercapnia in the pilot.

. FIG. 6 is a similar system to that of FIG. 5, although allowing for the use of environmental inputs to control pilot ventilation and/or the level of oxygen delivered to the pilot. Again, a microcontroller may be used to decide how the system componentry reacts according to the various inputs. For example, pilot ventilation and/or the level of oxygen delivered to the pilot may depend at least in part on g-forces experienced by pilot. The system may be more likely to increase ventilation and/or lower oxygen level output where high g-forces are present on the basis that hypercapnia is more likely (or is more likely to be of a higher severity) because g-forces can cause hypoventilation, atelectasis, inefficient gas exchange and in turn a V/Q mismatch. An oxygen sensor may be used as input to allow the system to determine how ventilation and/or oxygen level should be controlled: When in a low oxygen atmosphere it would be generally preferred to avoid lowering oxygen to the point where the result is to cause the pilot an episode of hypoxia. In a higher oxygen atmosphere such concern may be unwarranted. . Another input may be from an oxygen scheduler, which may be stored in electronic memory and used by the system to set parameters for ventilation and/or oxygen delivery based on altitude.

. FIG. 7 is a block diagram illustrating a different feedback loop which senses output parameters of exhalation and exhaled breath, and to use same to modulate oxygen level, gas flow control, peak rate control and diluent gas.

. FIG. 8 is a block diagram of a portion of the system illustrated in FIG. 5, comprising an auxiliary pump and backup air supply for use in the event of low or non-existent compressed air provided by the aircraft. As discussed elsewhere herein, many aircraft (such as the F-18S/H) can struggle under conditions of lower throttle to provide sufficient compressed air for the proper operation of the pilot breathing system. Under such conditions, the auxiliary subsystem shown in FIG. 7 actively pressurise the oxygen generator and also provide a high pressure source of diluent gas (ambient air) such that the system is able to effectively diluted the oxygen generator output under conditions of hypercapnia.

### EXAMPLES

### Example 1: Adverse Physiological Event Induced Under Actual Flight Conditions, and Resolution Thereof.

. This example is performed in a real high performance aircraft, with a pilot that reports previous UPEs e.g. F-18S/H

. A transcutaneous sensor tcPCO2 (with SpO2 option) (V-Sign™ sensor (SenTec AG, Switzerland)) is applied to the pilot’s skin at an area where reflectance SpO2 would function well (e.g. forehead, or against shoulder blades.)

. The pilot is instructed to fly with tcPCO2 / SpO2 sensor attached and information accumulated during the flight is logged into memory.

. The pilot is instructed to perform various high G-manoeuvres, and then run engine at “idle” revolution (causing OBOGS to malfunction) at agreed time intervals.

. The pilot is instructed not to “hyperventilate”, and to try to breathe “normally” as far as possible.

. Once the symptoms of an adverse physiological event are developed, the pilot is instructed to descend to below 10,000 ft, and to switch onto “emergency recovery gas mix” (being a diluted version of the gas previously breathed, the diluted gas mix acting to flush carbon dioxide from the pilot’s body) that is 20-30% oxygen and balance is helium. The instruction is to breathe through this recovery gas mix “full on” and extend exhalation 30% than “normal” (in order to assist with flushing of carbon dioxide) until symptoms are reversed. The reversal of symptoms proves that the cause of the symptoms was excess carbon dioxide in the blood (hypercapnia).

. A similar experiment could be performed a laboratory at sea level, by use of an “avmed” chamber having a pressure and oxygen regulator adjusted so as to mimic conditions in a high performance aircraft.

. The subject is instructed to breathe normally and perform some generic cognitive tasks, whilst their PaO2 / SpO2 is monitored by the same model tcPCO2.

. Since there is no G-atelectasis - the result may be “zero effect” of carbon dioxide retention.

. In order to simulate atelectasis and carbon dioxide retention resulting from flight conditions, in a second series of experiments admixing carbon dioxide in the range of 1-5% (effectively creating gas mix Carbogen). The above chamber trials (at sea level) are repeated whilst monitoring tcPCO2 and SpO2.

. Once the symptoms of an adverse physiological event are declared (by repeating a battery of neurocognitive tests) the subject is supplied with a recovery gas mix (20/80 Heliox gas mix) to flush carbon dioxide from the subject’s body. Extended exhalation may be implemented to hasten the flushing process.

. In the experiment tcPCO2 is dynamically monitored by the attending doctorsv/researchers. It is expected that symptoms are diminished because the symptoms are caused by hypercapnia, and the reduction of oxygen is the gas mixed delivered to the subject (with the attendant flushing out of carbon dioxide by the balancing diluent gas) reverses the hypercapnia-induced narcosis.

### Example 2: Adverse Physiological Event Resulting From G-induced Atelectasis, and Resolution Thereof.

. This example is performed with the subject in a pilot training centrifuge such as the ATFS-400 31 as provided by Environmental Technics Corporation (PA, USA). Such a centrifuge is capable of inducing atelectasis in a subject, and is the therefore proposed to be useful in studying to role of atelectasis in inducing hypercapnia and any accompanying adverse physiological event.

. The ATFS-400 31 has flight simulation capabilities, and in this Example is fitted with a simulated cockpit and computerised flight simulator allowing the subject to engage in flight simulation tasks whilst being exposed to significant g-forces.

. A transcutaneous sensor tcPCO2 (with SpO2 option) (V-Sign™ sensor (SenTec AG, Switzerland)) is applied to the pilot’s skin at an area where reflectance SpO2 would function well (e.g. forehead, or against shoulder blades.)

. The pilot enters the centrifuge, and is instructed to perform a simulated flight under intermittent periods of high g-forces as would be experienced in flight during acceleration and combat manoeuvres whilst breathing 100% oxygen. The tcPCO2 / SpO2 sensor remains attached and information accumulated during the simulated flight is logged into memory.

. The pilot is instructed not to “hyperventilate”, and to try to breathe “normally” as far as possible when a symptom of an adverse physiological event (including) atelectasis is experienced.

. Once symptoms of an adverse physiological event are developed (and detected by ongoing cognitive testing), the pilot is exposed to positive pressure ventilation of a Heliox gas mix by way of a close fitting breathing mask. The subject is instructed to breathe through this gas mix and extend exhalation 30% than “normal” (in order to assist with flushing of carbon dioxide) until symptoms are reversed. The reversal of symptoms proves that the cause of the adverse physiological event was hypercapnia secondary to g-induced atelectasis.

. A further study seeks to identify whether an adverse physiological event cause by hypercapnia can be reversed by increasing alveolar participation in the pilot’s lungs. Upon detection of hypercapnia, the ventilation pressure at the end of exhalation may be increased as discussed supra, such pressure acting to keep the lungs open and increase the number of alveoli actively participating in gas exchange.

### Example 3: Effect of Chest Restraint on tcPCO2 While Breathing Pure Oxygen

. A trial was conducted on four human subjects of mixed gender and age. Each subject was in good general health, having no reported respiratory or other medical condition.

. PCO2 for each subject was continuously monitored and recorded for the course of the experiment by a transcutaneous sensor tcPCO2 (with SpO2 option; V-Sign™ sensor; SenTec AG, Switzerland).

. Ventilatory movement of each subject’s chest was physically restrained in a first limb of the experiment and was unrestrained in a second limb.

. Initially each subject in each limb breathed atmospheric gas, and once a stable PCO2 reading was obtained, pure oxygen was supplied via a mask.

. Parameters of respiration were maintained as close as possible to that experienced in a military mask (i.e. noticeable resistance to breathing, exhalation into ambient, no rebreathing).

. Results are shown for subject “DN” in FIG. 9, demonstrating a substantial increase in tcPCO2 when his chest was restrained, and no increase when it was not. This shows that hypercapnia results from breathing 100% oxygen when normal ventilator movement of the chest is inhibited.

. Given that the subject’s breathing would have been more shallow with the chest restrained, carbon dioxide levels in the blood rose steadily in the presence of 100% oxygen due to a lessened ability to wash out the gas through the lungs. However, when 100% oxygen was turned off, PCO2 dropped significantly and almost returned to baseline. Thus, 100% oxygen promotes hypercapnia in that circumstance thereby leading to carbon dioxide narcosis. Cessation of 100% oxygen reverses the narcosis.

. By contrast, when the subject’s ventilatory movements were not suppressed, the lungs more readily washed out carbon dioxide. Accordingly, breathing 100% oxygen did not lead to any sustained increase in PCO2.

. In any of the Examples (or indeed any other embodiment of the invention described herein), the treatment for hypercapnia can be triggered automatically (by sensor(s) of appropriate physiological parameter(s)) or by the subject manually actuating the treatment upon self-detection of an adverse physiological event or some prodrome of an adverse physiological event.

. Those skilled in the art will appreciate that the invention described herein is susceptible to further variations and modifications other than those specifically described. It is understood that the invention comprises all such variations and modifications which fall within the spirit and scope of the present invention.

. While the invention has been disclosed in connection with the preferred embodiments shown and described in detail, various modifications and improvements thereon will become readily apparent to those skilled in the art.

. Accordingly, the spirit and scope of the present invention is not to be limited by the foregoing examples, but is to be understood in the broadest sense allowable by law.

1. A personal breathing system for use by a pilot in a military aircraft, the system comprising:
(a) a breathing mask capable of forming a seal about the pilot’s mouth at least, the seal being sufficient so as to allow control of pilot ventilation or an amount of oxygen breathed by the pilot, the breathing mask having a microphone associated therewith for voice communications from the pilot,
(b) a source of substantially pure oxygen gas in gaseous communication with the breathing mask, and
(c) a gas delivery regulator configured to (i) control pilot ventilation and/or (ii) limit or decrease an amount of carbon dioxide generated by the pilot by controlling the amount of oxygen gas breathed by the pilot so as to prevent, lessen or reverse hypercapnia.

2. (canceled)

3. The personal breathing system of claim 1, wherein pilot ventilation is controlled so as to increase exit of carbon dioxide from the pilot’s blood into the-air spaces of the lungs, the increase in exit being sufficient to prevent, lessen or reverse hypercapnia in the pilot.

4. The personal breathing system of claim 1, wherein pilot ventilation is controlled so as to increase expulsion of carbon dioxide from the-air spaces in the pilot’s lungs, the increase in expulsion being sufficient to prevent, lessen or reverse hypercapnia in the pilot.

5. The personal breathing system of claim 1, wherein pilot ventilation is controlled so as to correct a V/Q ratio mismatch in the pilot, where (V=ventilation and Q=perfusion).

6. (canceled)

7. The personal breathing system of claim 1, wherein pilot ventilation is controlled by reference to ventilation rate, ventilation pressure, breathing rate, positive end expiratory pressure, tidal volume, or FiO₂ (fraction of inspired oxygen).

8. (canceled)

9. (canceled)

10. (canceled)

11. The personal breathing system of claim 1, comprising a biofeedback subsystem configured to sense a pilot a parameter indicative of hypercapnia or likely impending hypercapnia, and to modulate ventilation of the pilot so as to prevent, lessen or reverse hypercapnia in the pilot.

12. (canceled)

13. (canceled)

14. The personal breathing system of claim 1, comprising an oxygen delivery element configured to deliver oxygen to the pilot, and the oxygen delivery element is configured to limit or decrease the amount of carbon dioxide generated by the pilot, the limit or decrease being sufficient so as to prevent, lessen or reverse hypercapnia in the pilot, the oxygen delivery element being further configured to deliver a first breathable gas or gas mixture having a first level of oxygen at a first time point to the pilot , and at a second time point delivering to the pilot a second breathable gas mixture having a second level of oxygen, the second level of oxygen being lower than the first level of oxygen, wherein the system is configured so as to be actuatable so as deliver the second gas mixture so as to prevent, lessen or reverse hypercapnia in the pilot.

15. The personal breathing system of claim 14, wherein the first level of oxygen is sufficiently high so as to induce hypercapnia in a pilot breathing the first breathable gas or gas mixture and the second level of oxygen is sufficiently low so as to prevent, lessen or reverse hypercapnia in a pilot having breathed the first gas or gas mixture at the first time point.

16. (canceled)

17. (canceled)

18. (canceled)

19. (canceled)

20. (canceled)

21. (canceled)

22. The personal breathing system of claim 14, comprising a biofeedback subsystem configured to sense a pilot a parameter indicative of hypercapnia or likely impending hypercapnia, and to lower the level of oxygen delivered to the pilot where hypercapnia likely impending or is detected.

23. (canceled)

24. The personal breathing system of claim 14, comprising a diluent gas source configured to deliver a diluent gas, and system is configured such that oxygen in the first gas or gas mixture is diluted with the diluent gas to provide the second gas mixture.

25. (canceled)

26. (canceled)

27. (canceled)

28. The personal breathing system of claim 1, wherein the gas delivery regulator comprises one or more data inputs and the regulator is configured to (i) control pilot ventilation and/or (ii) control the amount of oxygen breathed by the pilot, the control being sufficient so as to prevent, lessen or reverse hypercapnia in the pilot.

29. The personal breathing system of claim 28, comprising one or sensors in data communication with the one or more data inputs of the regulator, and wherein the one or more sensors is/are selected from the group consisting of: a barometer, an altimeter, an accelerometer, an oxygen sensor, a carbon dioxide sensor, and a contaminant sensor.

30. The personal breathing system of claim 29 comprising an electronic controller having program instructions configured to receive input data from the one or more sensors, transform the input data to output data, the output data being in data communication with the regulator, wherein the program instructions are configured to control pilot ventilation and/or the amount of oxygen breathed by the pilot so as to prevent, lessen or reverse hypercapnia in the pilot.

31. (canceled)

32. (canceled)

33. A method for treating or preventing an adverse physiological event in a pilot disposed in a military aircraft and receiving oxygen supplementation via a breathing mask, the method comprising:
(i) providing a breathing mask capable of forming a seal about the pilot’s mouth at least, the seal being sufficient so as to allow control of pilot ventilation or an amount of oxygen breathed by the pilot, the breathing mask having a microphone associated therewith for voice communications from the pilot,
(ii) facilitating removal of carbon dioxide generated the pilot by controlling pilot ventilation and/or (iii) limiting or decreasing an amount of carbon dioxide generated by the pilot by controlling the amount of oxygen breathed by the pilot, wherein the control of pilot ventilation and/or the control of the amount of oxygen breathed by the pilot is sufficient so as to prevent, lessen or reverse hypercapnia in the pilot.

34. The method of claim 33, wherein pilot ventilation is controlled so as to increase exit of carbon dioxide from the pilot’s blood into air spaces of the lungs, the increase in exit being sufficient to prevent, lessen or reverse hypercapnia in the pilot.

35. The method of claim 33, wherein pilot ventilation is controlled so as to increase expulsion of carbon dioxide from air spaces in the pilot’s lungs, the increase in expulsion being sufficient to prevent, lessen or reverse hypercapnia in the pilot.

36. The method of claim 33, wherein pilot ventilation is controlled so as to correct a V/Q ratio mismatch in the pilot, where V=ventilation and Q=perfusion.

37. The method of claim 36, wherein pilot ventilation is controlled to increase the V/Q ratio, the increase being sufficient to prevent, lessen or reverse hypercapnia in the pilot.

38. The method of claim 33, wherein pilot ventilation is controlled by reference to ventilation rate, ventilation pressure, breathing rate, positive end expiratory pressure tidal volume, or FiO₂ (fraction of inspired oxygen).

39. The method of claim 38, wherein ventilation rate is increased, or tidal volume is increased or ventilation pressure is increased, the increase being sufficient to prevent, lessen or reverse hypercapnia in the pilot.

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)
